(19) United States
(12) Patent Application Publication
(10) Pub. No.: US 2018/0263568 A1
(43) Pub. Date: Sep. 20, 2018

(54) Systems and Methods for Clinical Image Classification

(71) Applicant: The Board of Trustees of the Leland Stanford Junior University, Stanford, CA (US)

(72) Inventors: Darvin Yi, Menlo Park, CA (US); Timothy Chan Chang, Mountain View, CA (US); Joseph Chihping Liao, Stanford, CA (US); Daniel L. Rubin, Palo Alto, CA (US)

(73) Assignee: The Board of Trustees of the Leland Stanford Junior University, Stanford, CA (US)

(21) Appl. No.: 15/917,494

(22) Filed: Mar. 9, 2018

Related U.S. Application Data
(60) Provisional application No. 62/469,405, filed on Mar. 9, 2017.
(60) Provisional application No. 62/469,441, filed on Mar. 9, 2017.
(60) Provisional application No. 62/483,231, filed on Apr. 7, 2017.

(51) Int. Cl. A61B 5/00; G06T 7/00; A61B 1/00; A61B 5/20; A61B 1/04

(52) U.S. Cl. CPC A61B 5/7267; G06T 7/0016; A61B 5/0068; A61B 1/00172; G06T 2207/30096; A61B 1/04; A61B 5/0084; G06T 2207/20081; G06T 2207/30024; A61B 5/20

(57) ABSTRACT

Systems and methods for performing image processing in accordance with embodiments of the invention are illustrated. One embodiment includes an imaging system including at least one processor, an input/output interface in communication with a medical imaging device, a display in communication with the processor, and a memory in communication with the processor, including image data obtained from a medical imaging device, where the image data describes at least one image describing at least one region of a patient's body, and an image processing application, where the image processing application directs the processor to preprocess the image data, identify pathological features within the preprocessed image data, calculate the likelihood that the at least one region described by the at least one image is afflicted by a disease, and provide a disease classification substantially instantaneously describing the disease and the likelihood of the disease being present in the region via the display.

### CROSS-REFERENCE TO RELATED APPLICATIONS

The current application claims priority under 35 U.S.C. § 119(e) to U.S. Provisional Application Ser. No. 62/469,405 entitled “Dynamic and Automated Classification of Bladder Cancer Using Deep Learning on Streaming Confocal Laser Endomicroscopy Images” to Chang et al., filed Mar. 9, 2017, to U.S. Provisional Application Ser. No. 62/469,441 entitled “Dynamic and Automated Classification of Bladder Cancer Using Deep Learning on Streaming Confocal Laser Endomicroscopy Images” to Chang et al., filed Mar. 9, 2017, and to U.S. Provisional Application Ser. No. 62/483,231 entitled “System and Method for Automated Classification of Medical Images Using Convolutional Neural Networks” to Yi et al., filed Apr. 7, 2017. U.S. Provisional Application Ser. Nos. 62/469,405, 62/469,441, and 62/483,231 are hereby incorporated by reference in its entirety.

### FIELD OF THE INVENTION

This invention generally relates to the automated diagnosis of disease using deep learning. More particularly, this invention relates to the near real-time classification of structures within medical images of cellular structure using convolutional neural networks.

### BACKGROUND

In the medical field, various imaging technologies are used to evaluate and diagnose conditions of the human body. Endoscopies have long been used in the medical field for visual examination of the interiors of body cavities and hollow organs. A medical professional may use an endoscope to investigate symptoms, confirm a diagnosis, and/or provide treatment. An endoscope is an instrument with a rigid or flexible tube, a lighting system to illuminate the organ, and an imaging system to transmit images to the viewer. Various types of endoscopes are available for examination of different organs, such as a cystoscope for the lower urinary tract, an enteroscope for the small intestine, a bronchoscope for the lower respiratory tract, and many others. The endoscope is typically inserted directly into the organ, and may be fitted with a further apparatus for examination or retrieval of tissue. Modern endoscopes are often videoscopes, transmitting images from a camera to a screen for real-time viewing by the health professional. The procedure may then be reviewed through video playback, or condensed into a few still images with notes and drawings. Data may be captured via the use of various modalities that be deployed endoscopically, including, but not limited to, standard white light endoscopy (WLE), fluorescence, spectroscopy, confocal laser endomicroscopy (CLE) and optical coherence tomography (OCT).

Bladder cancer is one condition for which diagnosis typically involves the use of cystoscopy (endoscopy of the bladder). As the fifth most common cancer in the U.S., bladder cancer presents a high recurrence rate. With surveillance endoscopies being recommended up to every three months, bladder cancer is estimated to have the greatest per-patient lifetime cost of all cancers.

Standard cystoscopy employs the white light endoscopic modality (WLE). Certain challenges of using WLE include multi-focality of the bladder tumors, differentiation of neoplastic tissue from benign and inflammatory lesions, co-existence of papillary and flat lesions, determination of tumor boundaries, and quality of optical imaging. Currently, the standard for cancer diagnosis is through evaluation by pathology. This process includes tissue fixation, staining and then evaluation by a pathologist, and may take up to a week.

Optical biopsy technologies such as CLE and OCT provide high-resolution, micron-scale imaging during a procedure. Placing the CLE probe against organ tissue, clinicians may perform an “optical biopsy” in real time during the endoscopy. The high-resolution, dynamic, sub-surface imaging of CLE has a proven track record in gastrointestinal and pulmonary applications, such as in the diagnosis of colonic dysplasia and Barrett's esophagus. Alternatively, ultrasound imaging uses sound waves of an ultrasonic frequency to perform medical imaging. Ultrasound technology may be used visualize muscles, tendons, and internal organs, and evaluate their structures in real time.

### SUMMARY OF THE INVENTION

Systems and methods for performing image processing in accordance with embodiments of the invention are illustrated. One embodiment includes an imaging system including at least one processor, an input/output interface in communication with a medical imaging device, a display in communication with the processor, and a memory in communication with the processor, including image data obtained from a medical imaging device, where the image data describes at least one image describing at least one region of a patient's body, and an image processing application, where the image processing application directs the processor to preprocess the image data, identify pathological features within the preprocessed image data, calculate the likelihood that the at least one region described by the at least one image is afflicted by a disease, and provide a disease classification substantially instantaneously describing the disease and the likelihood of the disease being present in the region via the display.

In another embodiment, the medical imaging device is a confocal laser endoscope.

In a further embodiment, the components of the imaging system are integrated into a single imaging device.

In still another embodiment, the image data describes a video including at least two sequential frames describing at least one region of a patient's body.

In a still further embodiment, the image processing application further directs the processor to preprocess a first frame in the video, identify pathological features within the first preprocessed frame, calculate the likelihood of disease in the region described by the first frame, provide a disease classification substantially instantaneously describing the disease and the likelihood of the disease being present in the region via the display, preprocess a second frame in the video, identify pathological features within the second preprocessed frame, calculate the likelihood of disease in the region described by the second frame, and update the disease classification substantially instantaneously based on the second frame.

In yet another embodiment, the image processing application further directs the processor to provide a disease classification for the region described by all of the frames in the video via the display.

In a yet further embodiment, the region of the patient's body is the bladder and the disease is a type of bladder disease selected from the group consisting of high grade cancer, low grade cancer, carcinoma in situ, and inflammation.

In another additional embodiment, to preprocess the image data, the image processing application further directs the processor to standardize the resolution of each frame, and center each frame.

In a further additional embodiment, to identify pathological features within the preprocessed image data, the image processing application further directs the processor to provide images to a convolutional neural network, where the convolutional neural network is trained by providing classified images of diseased features.

In another embodiment again, to calculate the likelihood of disease in a region, the image processing application further directs the processor to obtain a probability score from the convolutional neural network describing the likelihood that the convolutional neural network has correctly identified a disease within the frame.

In a further embodiment again, the pathological features are structural features of bladder cells associated with any of normal cells, high grade cancer cells, low grade cancer cells, carcinoma in situ cells, and inflammatory cells.

In still yet another embodiment, a method for providing a substantially instantaneous disease classification based on image data includes obtaining image data from a medical imaging device, where the image data describes at least one image describing at least one region of patient's body using an image processing server system, wherein the image processing server system includes at least one processor, an input/output interface in communication with the medical imaging device and the processor, a display in communication with the processor, and a memory in communication with the processor, where the memory is configured to store the image data, preprocessing the image data using an image processing server system, identifying pathological features within the preprocessed image data, calculating the likelihood that the at least one region described by the at least one image is afflicted by a disease, providing a disease classification substantially instantaneously describing the disease and the likelihood of the disease being present in the region via the display.

In a still yet further embodiment, the medical imaging device is a confocal laser endoscope.

In still another additional embodiment, the image data describes a video including at least two sequential frames describing at least one region of a patient's body.

In a still further additional embodiment, the disease classification is provided based on a first frame in the at least two sequential frames, and updating the disease classification based on the second frame.

In still another embodiment again, the method further includes providing a disease classification for the region described by all of the frames in the video via the display.

In a still further embodiment again, the region of the patient's body is the bladder and the disease is a type of bladder disease selected from the group consisting of high grade cancer, low grade cancer, carcinoma in situ, and inflammation.

In yet another additional embodiment, preprocessing the image data includes, standardizing the resolution of each frame, and centering each frame.

In a yet further additional embodiment, identifying pathological features within the preprocessed image data includes providing images to a convolutional neural network, where the convolutional neural network is trained by providing classified images of diseased features.

In yet another embodiment again, calculating the likelihood of disease in a region includes obtaining a probability score from the convolutional neural network describing the likelihood that the convolutional neural network has correctly identified a diseased structure within the frame.

In a yet further embodiment again, the pathological features are structural features of bladder cells associated with any of normal cells, high grade cancer cells, low grade cancer cells, carcinoma in situ cells, and inflammatory cells.

In another additional embodiment again, a method for providing a substantially instantaneous disease classification based on image data includes obtaining image data from a confocal laser endoscope, where the image data describes at least one video including at least a first frame and a second frame describing at least a first region and a second region of a patient's bladder, using an image processing server system, wherein the image processing server system includes, at least one processor, an input/output interface in communication with the confocal laser endoscope, a display in communication with the processor, and a memory in communication with the processor, where the memory is configured to store the image data, preprocessing the first frame and the second frame using an image processing server system, identifying a first set of pathological features within the first preprocessed frame using a convolutional neural network, where the convolutional neural network is trained by providing it with ground truth annotated images of various types of bladder cancer, calculating the likelihood that the bladder is afflicted by a type of bladder cancer based on the first set of pathological features, providing a disease classification substantially instantaneously describing the type of bladder cancer and the likelihood of the type of bladder cancer being present in the region via the display, identifying a second set of pathological features within the second preprocessed frame using a convolutional neural network, calculating the likelihood that the bladder is afflicted by a type of bladder cancer based on the second frame, and providing an updated disease classification based on the likelihoods calculated from the first and second preprocessed frames.

In a further additional embodiment again, an imaging system including at least one processor, an input/output interface in communication with a confocal laser endoscope and the processor, a display in communication with the processor, and a memory in communication with the processor, including image data obtained from a medical imaging device, where the image data describes at least one video including at least a first frame and a second frame describing at least a first region and a second region of a patient's bladder, and an image processing application, where the image processing application directs the processor to preprocess the first frame and the second frame, identify a first set of pathological features within the first preprocessed frame using a convolutional neural network, where the convolutional neural network is trained by providing it with ground truth annotated images of various types of bladder cancer, calculate the likelihood that the bladder is afflicted by a type of bladder cancer based on the first set of pathological features, provide a disease classification substantially instantaneously describing the type of bladder cancer and the likelihood of the type of bladder cancer being present in the region via the display, identify a second set of pathological features within the second preprocessed frame using a convolutional neural network, calculate the likelihood that the bladder is afflicted by a type of bladder cancer based on the second frame, and provide an updated disease classification based on the likelihoods calculated from the first and second preprocessed frames.

In still yet another additional embodiment, the image processing application further directs the processor to alert a user that a pathological feature has been detected using the display.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure

### BRIEF DESCRIPTION OF THE DRAWINGS

FIG. 1 illustrates a network diagram of an image processing system in accordance with an embodiment of the invention.

FIG. 2 illustrates an image processing server system in accordance with an embodiment of the invention.

FIG. 3 is a flow chart illustrating a method for providing disease classification based on image data in accordance with an embodiment of the invention.

FIG. 4 is a flow chart illustrating a method for providing disease classification of bladder cancer based on CLE image data in accordance with an embodiment of the invention.

FIG. 5 is an illustration of a normal cell feature and associated disease classification in accordance with an embodiment of the invention.

FIG. 6 is an illustration of a cancerous cell feature and associated disease classification in accordance with an embodiment of the invention.

FIG. 7 is an illustration of an inflamed cell feature and associated disease classification in accordance with an embodiment of the invention.

### DETAILED DESCRIPTION

Currently, deep learning techniques are revolutionizing the field of computer science. Deep learning techniques such as, but not limited to, artificial neural networks and their variants have led to leaps forward in various computational applications such as machine vision, audio processing, and natural language processing. While several attempts have been made to apply neural network technology to medical diagnostics in the field of bioinformatics, many of the data sets used are vast quantities of genetic data and/or a set of biomarkers. Indeed, it is image processing that has seen some of the most substantial success in the utilization of deep learning techniques. As such, the application of image processing neural networks to the medical field represents a shortcut to systems and methods for the disease classification based on phenotype, which can be used by a medical professional in the development of their prognosis and/or diagnosis.

Furthermore, many conventional image processing based diagnostic methods do not operate in real time. Images obtained by imaging methods such as endoscopies or ultrasound are stored and read post-hoc by radiologists or other specialists. In some cases, when a suspicious lesion is encountered during a procedure, whether open, laparoscopic, or endoscopic, the standard of care is to remove or biopsy the tissue and send it to pathology. The tissue is fixed, stained, and read by the pathologist. The entire process may be time consuming and can take up to a week. While the time to perform these tests is long, the “gold standard” for diagnosing many diseases such as, but not limited to, various types, grades, and stages of cancers has traditionally involved a histological and/or cytological analysis. When analysis and diagnosis is performed after the initial investigatory procedure, the diseased region must be relocated during any required secondary procedures which can be difficult depending on the type of imaging data obtained.

As such, there are situations in the clinical setting such as in the Emergency Room or in the operating room where having expertise in the interpretation of the imaging at the time of image acquisition may be beneficial. For example, if a lesion is noted on either an ultrasound or endoscopy, the clinician can perform a biopsy at the time of image acquisition. In addition, if a potentially suspicious area is seen while acquiring the ultrasound our endoscopy images, the clinician can more carefully image the suspicious area and confirm whether it is truly an abnormality or an artifact. If a disease classification is obtained in real time, the medical professional can take immediate action without the need for a secondary procedure or relocating the region at a later date. However, the obtained image data from the procedure can be difficult for even a trained medical professional to parse in real time. Indeed, many imaging methods, such as CLE, show the imaged biological structure at the cellular level, and there may be on the order of tens of billions to trillions of cells that make up any particular organ.

Further, since the accuracy in interpreting the images depends on the operating clinician's experience and overall ability that can vary significantly, a smart tool that provides automated classification can augment the value of the imaging itself so that the clinician can make real-time decisions. Again, this can be applied to any modality that provides real-time imaging, including but not limited to ultrasound and endoscopy. Methods according to certain embodiments of the invention, capture real-time imaging, utilize CNNs for image classification, and provide real-time, frame-by-frame feedback to the operator through its application on an endomicroscopy system.

Turning now to the drawings, systems and methods for imaging systems involving the real-time, automated classification of features within medical images to provide clinical disease classifications are illustrated. When discussing real-time actions in the computing context, one of ordinary skill in the art would appreciate that “real-time” refers to a time scale on the order of seconds or below, also referred to as “substantially real-time”. In numerous embodiments, processes described below operate on the order of approximately one second or below. However, the lag time can be reduced by increasing the computing power of the system, and may be higher with low computing power. In several embodiments of the invention, the features are detected within images of the cellular structure and tissue microarchitecture of a patient. According to some embodiments of the invention, abnormalities may be identified. As is discussed further below, systems and methods in accordance with various embodiments of the invention can be utilized to provide clinical pathology information in a variety of medical imaging contexts in real time. While systems and methods described herein can be adapted to any number of different pathologies, the following discussion will focus on identification and classification of bladder cancers as exemplary embodiments in accordance with various applications of the invention.

### Image Processing Systems

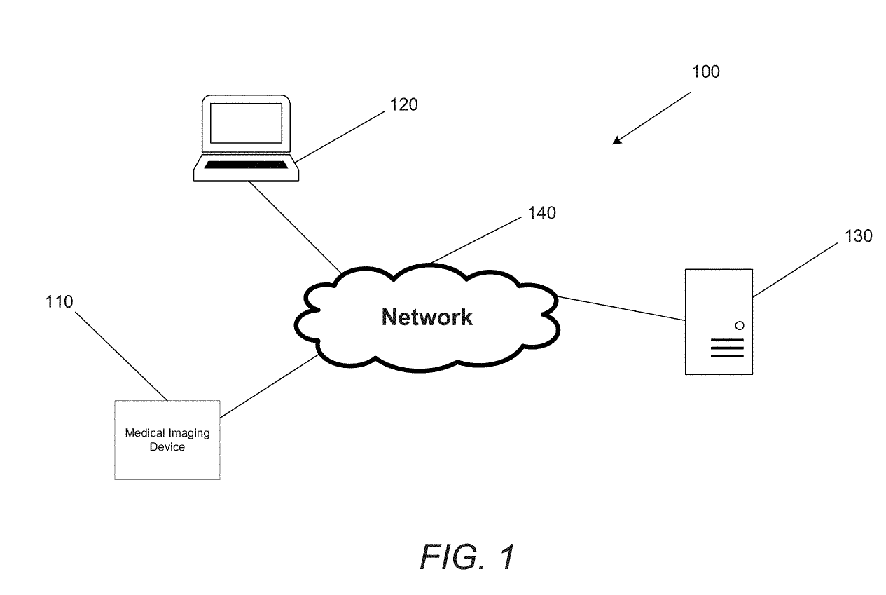

In many embodiments, image processing systems obtain image data from a patient using a medical imaging device and process the images in real time to produce a disease classification. Turning now to FIG. 1, a network diagram of an image processing system in accordance with an embodiment of the invention is illustrated. In many embodiments, image processing system 100 utilizes a medical imaging device 110. Medical imaging devices can be any tool that obtains image data describing the features of a region of the body at a cellular level such as, but not limited to, a CLE or a WLE. System 100 includes an interface device 120 and an image processing server system 130. Interface devices are any device capable of displaying diagnostic results. In numerous embodiments, interface devices can be used to control the movement of medical imaging devices. Interface devices can be implemented using personal computers, tablet computers, smart phones, or any other computing device capable of displaying diagnostic results. In a variety of embodiments, image processing server systems enable image processing applications to generate disease classifications from image data. Image processing server systems can be implemented using one or more servers, personal computers, smart phones, tablet computers, or any other computing device capable of running image processing applications. In numerous embodiments, image processing server systems and interface devices are implemented using the same hardware.

Image processing system 100 further includes a network 140. Network 140 is any network capable of transferring data between medical imaging devices, interface devices, and image processing server systems. Networks can be intranets, the Internet, local area networks, wide area networks, or any other network capable of transmitting data between components of the system. Networks can further be wired, wireless, or a combination of wired and wireless.

While a specific system architecture in accordance with an embodiment of the invention is illustrated in FIG. 1, any number of system architectures can be utilized as appropriate to the requirements of specific applications of embodiments of the invention. For example, image processing systems in accordance with embodiments of the invention could be implemented on a single medical imaging device with added processing capabilities. Implementations of image processing servers are described below.

### Image Processing Server Systems

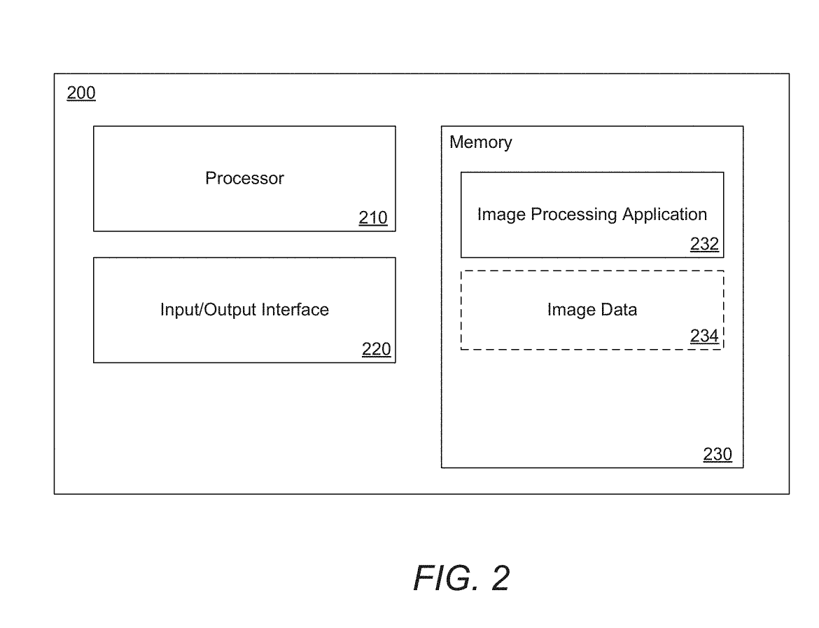

Image processing server systems are capable of running image processing applications to generate disease classifications from image data. Turning now to FIG. 2, a conceptual illustration of an image processing server in accordance with an embodiment of the invention is illustrated. Image processing server system 200 includes a processor 210. Processors can be any logic unit capable of processing data such as, but not limited to, central processing units, graphical processing units, microprocessors, parallel processing engines, or any other type of processor as appropriate to the requirements of specific applications of embodiments of the invention. Image processing server system further includes an input/output interface 220 and memory 230. Input/output interfaces are capable of transferring data between the image processing server, interface devices, and medical imaging devices. Memory can be implemented using any combination of volatile and/or non-volatile memory, including, but not limited to, random access memory, read-only memory, hard disk drives, solid-state drives, flash memory, or any other memory format as appropriate to the requirements of specific applications of embodiments of the invention.

Memory 230 contains an image processing application 232 capable of directing the processor to perform image processing processes on image data to produce at least one disease classification in accordance with an embodiment of the invention. Memory 230 at times contains image data 234 which is processed by the processor in accordance with the processes described by the image processing application. Image data can be any type of data obtained from a medical imaging device including, but not limited to, a single image, a set of images stored as separate image files, or a set of images stored as a single image file, for example a video file. A single image taken at a particular time by a medical imaging device is called a frame. Video files are made up of temporally ordered sequences of frames. However, image data can be in any format or structured in any way as appropriate to the requirements of specific applications of embodiments of the invention.

While a specific image processing server system is described above with respect to FIG. 2, any number of architectures for image processing server systems, including those distributed across multiple computing platforms, can be utilized as appropriate to the requirements of specific applications of embodiments of the invention. A number of image processing processes in accordance with various embodiments of the invention are described below.

### Generating Disease Classifications Based On Image Data

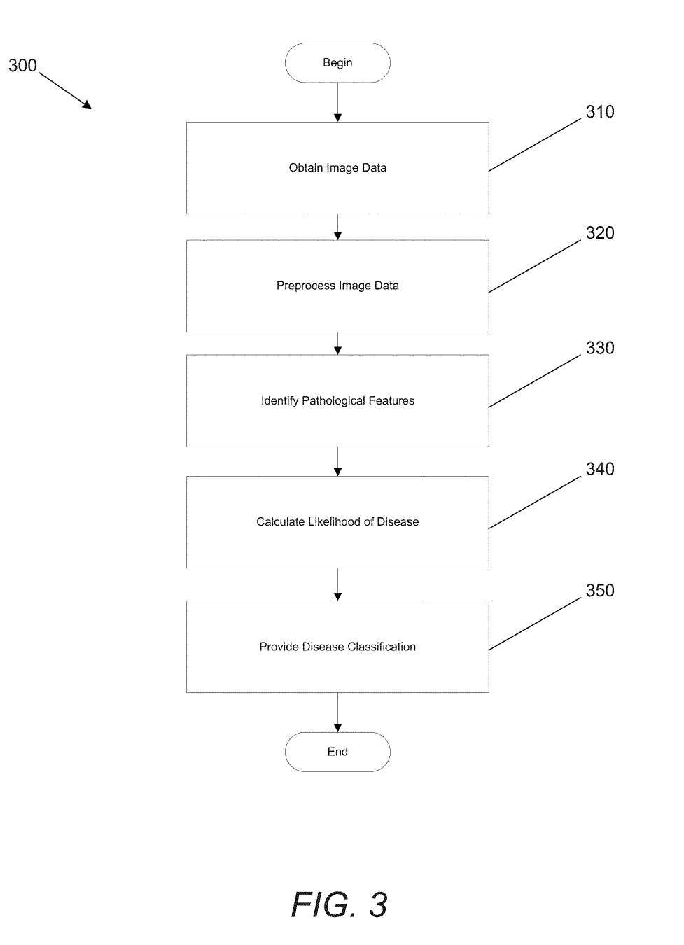

Image processing processes can generate disease classifications that suggest one or more conditions that may afflict a patient based on image data obtained from imaging their body. In numerous embodiments, image processing processes can provide actionable disease classifications that result in a specific treatment being applied to the patient. Real-time disease classifications provided by the image processing processes can enable medical professionals to take immediate action when medical instruments are already focused on the identified diseased region. Turning now to FIG. 3, an image processing process to generate a disease classification based on image data in accordance with an embodiment of the invention is illustrated.

Process 300 includes obtaining (310) image data. In numerous embodiments, image data is obtained from a medical imaging device. Image data is preprocessed (320). In a variety of embodiments, image data is preprocessed by selecting single frames from a video sequence of images. In several embodiments, frames are preprocessed as they are obtained. However, in many embodiments, not all frames are preprocessed. Single frames can be selected at pre-determined time intervals and/or random time intervals for preprocessing. However, in a variety of embodiments, every frame in a video sequence is processed. Frames where the medical imaging device was not imaging tissue can be trimmed to reduce processing time and/or bad data input. However, not all frames with bad data are necessarily trimmed. For example, a threshold can be set whereby frames with a set number of pixels as a single color can be trimmed (e.g. all white, all black, static patterns due to heavy noise). Preprocessing can further include standardizing the resolution of every image, randomly rotating images, standardizing the shape of every image, centering every image, and/or any other preprocessing set as appropriate to the requirements of specific applications of embodiments of the invention. In a variety of embodiments, the standardized dimensions are 512×510 pixels, however, any resolution can be used as appropriate to the requirements of specific applications of embodiments of the invention. For example, images can be preprocessed such that the image is generally zero-centered and has a standard deviation of pixel values around 1. In a variety of embodiments, images can be saved as 8-bit portable network graphic images, and 128 pixels can be subtracted from the pixel range of 0-225, and that value can be divided by 32 with a conversion to single precision floats. In this way, every image is unchanged relative to the other images, but the pixel values are in a more statistically safe range. However, any number of processing techniques can be used to generate statistically safe image size. Further, any file format type can be used as appropriate to the requirements of specific applications of embodiments of the invention.

Process 300 further includes identifying (330) pathological features. In numerous embodiments, identifying pathological features is achieved by feeding preprocessed image data into a CNN. In many embodiments, the CNN is trained using a data set consisting of a number of annotated images describing the different ground truth classifications of features within each image in the data set. In this way, the CNNs are trained via machine learning processes to provide clinical pathology information. Many embodiments of the invention utilize classes of CNNs that are specifically chosen to enable efficient computation to provide real-time clinical pathology information, including, but not limited to, the identification of different grades and/or of a disease (e.g. a grade and/or stage of cancer). In certain embodiments, one or more CNNs are trained to enable the classification of image data obtained during a procedure into a number of medically relevant categories. These classifications can be expressed in terms of the likelihood with which each image corresponds to each of the categories. According to certain embodiments of the invention, the CNNs used are a low-bias methods that may search a large parameter space with minimal rules, with possibly the only goal of maximizing accuracy. The network may receive only the input images and the corresponding disease classifications, from which the network learns how to maximize accuracy. In this way, CNNs utilized in accordance with many embodiments of the invention are able to learn from image data that provide clinical pathology information that is typically obtained through tools of chemistry, clinical microbiology, hematology and/or molecular pathology.

Furthermore, appropriately selected CNNs can provide comparable clinical pathology information from image data in real time. In a variety of embodiments, the CNN architecture is GoogLeNet produced by Google LLC of Mountain View, Calif. However any number of CNN architectures can be utilized. By running the trained CNN on the preprocessed image data, the CNN is able to identify pathological features which it was initially shown in real-time. In numerous embodiments, the CNN further outputs a probability score indicating the likelihood that a particular feature is identified within the image. In numerous embodiments, probability scores are influenced by temporally adjacent frames. Features generally appear over regions of a diseased organ. By accounting for temporally adjacent frames, false positives can be reduced because temporally adjacent frames should on average contain similar structures. Similarly, positively identifying features in consecutive frames increases the likelihood of a correct identification. However, it is not necessary to have “look-back” functionality in order for the processes described to function.

A likelihood of disease can be calculated (340). In many embodiments, the overall likelihood of a disease state is calculated by averaging the probability scores for each image in the image data. Likelihood of disease can be calculated at both a general and a specific level. For example, a general level classification could indicate whether or not any disease is present. A specific level classification could indicate the specific type of disease and/or the structures present. Based on the calculated likelihoods of disease, a disease classification can be provided (350). In many embodiments, disease classifications are updated in real time as more image data is received. In a variety of embodiments, disease classifications are provided on a per-frame basis. However in numerous embodiments, disease classifications are provided directed to the entire imaged structure based on multiple frames.

As disease classifications can be provided in real time, they can further include indications to the medical professional to look more closely at the location currently being imaged by the medical imaging device. In a variety of embodiments, the disease classification can include a treatment option. Treatment steps can include additional diagnostic steps to confirm the disease classification such as, but not limited to, a biopsy and/or any other diagnostic test as appropriate to the requirements of specific applications of embodiments of the invention. Treatment options can be identified by matching the diagnosed disease with a set of treatments in a treatment database keyed by disease. For example, identified lesions may be treated by stitching, application of a drug, cauterization, resection, and/or any other preventative or reparative treatment as appropriate to treat the identified disease. Furthermore, alerts can be issued in substantially real-time to indicate to a user that they should pay particular attention to the region currently being imaged. Alerts can be issued visually via display and/or a light, audibly using a speaker, or by any combination thereof. In numerous embodiments, alerts can be associated with a disease classification. In a variety of embodiments, alerts can indicate a need to re-image an area to obtain more and/or better image data.

In numerous embodiments, the disease classification is provided on a per-frame basis as the medical imaging device is imaging the patient. In a variety of embodiments, an overall disease classification for the organ being imaged is provided that is consistently being updated as new frames are processed. For example, in numerous embodiments, preprocessing of image data is a continuous process where frames are preprocessed as they are captured by a medical imaging device. A CNN can be applied to preprocessed frames as they are generated such that live disease classifications on a per-frame basis can be provided to a medical practitioner.

While the process above is generally directed to the classification and/or localization of any number of diseases using image data, specific implementations for particular types of image data and/or particular classes of diseases can be performed by image processing systems as well. A specific implementation for diagnosing bladder cancer based on CLE image data is described below.

### Automatically Diagnosing Bladder Cancer Using Image Data

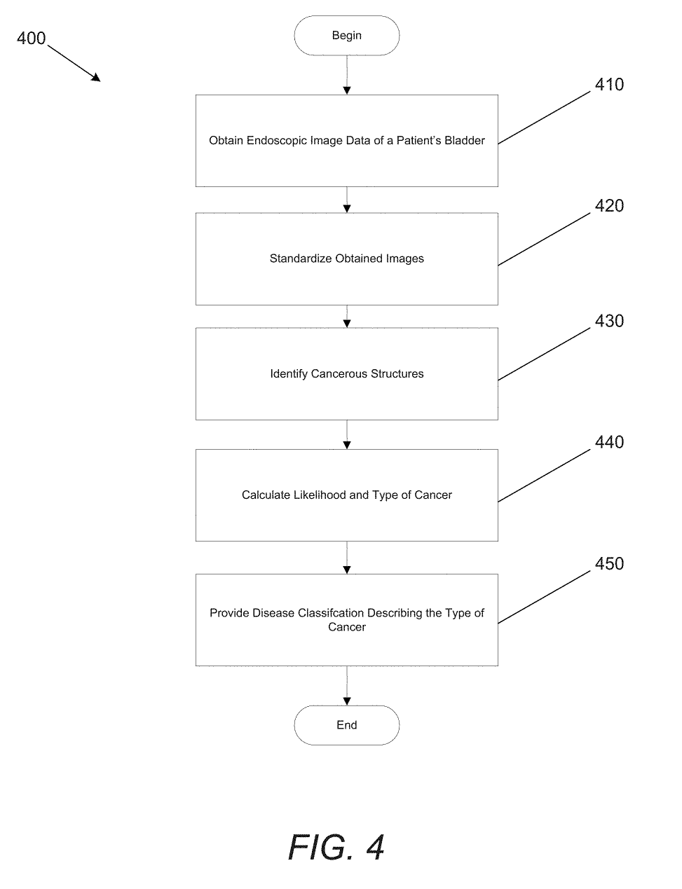

As noted above, immediate feedback during an internal procedure such as a bladder endoscopy would be invaluable to medical practitioners. Further, a system which enables even practitioners who are untrained in reading endoscopy images to issue disease classifications to a patient with equal or greater accuracy than a trained human professional would reduce burden on hospitals and enable quicker reaction and immediate follow-up procedures, reducing the stress on the patient's body. In addition, a system that provides feedback as to likely disease classifications could help medical professionals to better detect disease by pinpointing suspicious regions in the image, prompting the professional to confirm the abnormality by doing additional focused imaging in the suspicious area(s). Image processing processes can include the automatic diagnosing of bladder cancers using image data obtained from CLE and/or WLE devices. Turning now to FIG. 4, a process for diagnosing bladder cancer based on image data in accordance with an embodiment of the invention is described below.

Process 400 includes obtaining (410) endoscopic image data of a patient's bladder. The current standard for visualizing bladder cancers is through WLE. Trained human urologists have experimentally been able to diagnose bladder conditions using WLE with 84% accuracy (86% sensitivity, 80% specificity). Similarly, trained human urologists have experimentally been able to diagnose bladder conditions using CLE with 79% accuracy (77% sensitivity, 82% specificity). Systems and methods described herein are able to disease classifications these conditions using CLE imaging with 87% accuracy (79% sensitivity, 90% specificity), thereby outperforming trained human counterparts. Further, humans given multimodal imaging data, e.g. both CLE images and conventional WLE images have shown improved performance.

In numerous embodiments, the endoscopic image data is a video sequence obtained by a CLE such as a Cellvizio system produced by Manua Kea Technologies of Paris, France. However in many embodiments, the endoscopic image data is a video sequence obtained by a WLE or multiple video sequences obtained by a CLE and a WLE respectively. Indeed, endoscopic image data can be obtained by any endoscopic imaging method, or contain multimodal images.

The obtained images can be standardized (420) using methods similar to those described above. In many embodiments, image processing systems can be overlaid onto existing CLE imaging systems by capturing the video feed from the display of the CLE imaging system by using screen capture software. The video feed can be captured in a variety of ways, including, but not limited to, utilizing video capture hardware (e.g. video capture cards) and/or software on the output of CLE systems. In this way, existing systems do not need to be replaced with completely integrated image processing systems. If screen capture tools are utilized, preprocessing can further include identifying the region of the screen that contains the image data from the CLE, cropping to that region, and performing additional preprocessing techniques on the cropped image data. However, image processing systems can be built directly into medical imaging devices such as CLEs such that the output of a conventional CLE system is augmented with real-time disease classification feedback described below.

Cancerous structures can be identified (430) using a CNN in a fashion similar to those described above. Structural features can be utilized to not only detect the presence of cancerous regions, but classify the specific type of cancer. Bladder cells can be categorized into four main categories: normal, low grade (LG), and high grade (HG), and inflammatory, with a fifth classification of carcinoma in situ (CIS) as a subclass of HG. These categories can be defined structurally as follows. Normal bladder cells are flat, organized, monomorphic in nature, and have clear and distinct cell borders. LG cancer cells are organized and monomorphic, but configured as a papillary structure with a fibrovascular stalk. HG cancer cells are usually papillary but are disorganized, pleomorphic cells with indistinct cell borders. CIS cells are similar to HG cells but are flat instead of papillary. Inflamed cells are small, clustered non-cancerous, inflammatory cells with distinct cell borders. In some embodiments, the CNN is trained with a dataset containing images of bladder cancer annotated with structural classifications corresponding to various types of bladder cancer.

However, as bladder cancer cells can be visually distinct as described above, in numerous embodiments, the CNN is trained with a dataset containing images of bladder cancer annotated with classifications including normal, LG, HG, CIS, and inflammatory without associated structural information. In this way, CNNs can be trained to identify particular structural features associated with the classifications such as those described above and/or any other structural feature that may or may not be readily apparent to the human eye as appropriate to the requirements of specific applications of embodiments of the invention. In a variety of embodiments, a secondary calculation is performed to identify the type of cancer based off of only the structural features identified. However, in many embodiments, the identification of structural features are utilized within the CNN to identify a cancer classification. As such, systems and methods described herein can not only identify whether or not a region is cancerous, but also provide cancer typing and/or staging.

In numerous embodiments, the CNN outputs a probability score for each feature identified per image. In many embodiments, the CNN outputs probability scores for each of the normal, LG, HG, CIS, and inflammatory categories per image. A likelihood and type of cancer for the entire video sequence in the image data can be calculated (440) by averaging the probability scores for each frame. A bladder cancer disease classification can be provided (450) based on the calculated likelihoods. In numerous embodiments, the disease classification can indicate the presence of no cancer, or at least one type of cancer. Further, in a variety of embodiments, the image data can be used to construct a 3D model of the bladder, and the 3D model can be annotated with identified cancerous regions using systems and methods described in U.S. patent application Ser. No. 15/233,856 titled “3D Reconstruction and Registration of Endoscopic Data” which is hereby incorporated by reference in its entirety. In a variety of embodiments, the process further includes recommending and providing a treatment to the patient based on the disease classification.

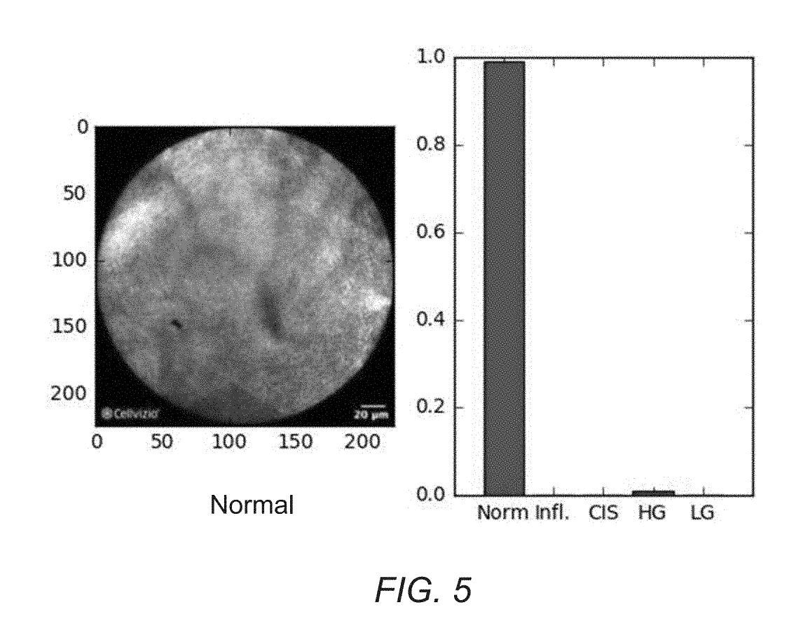

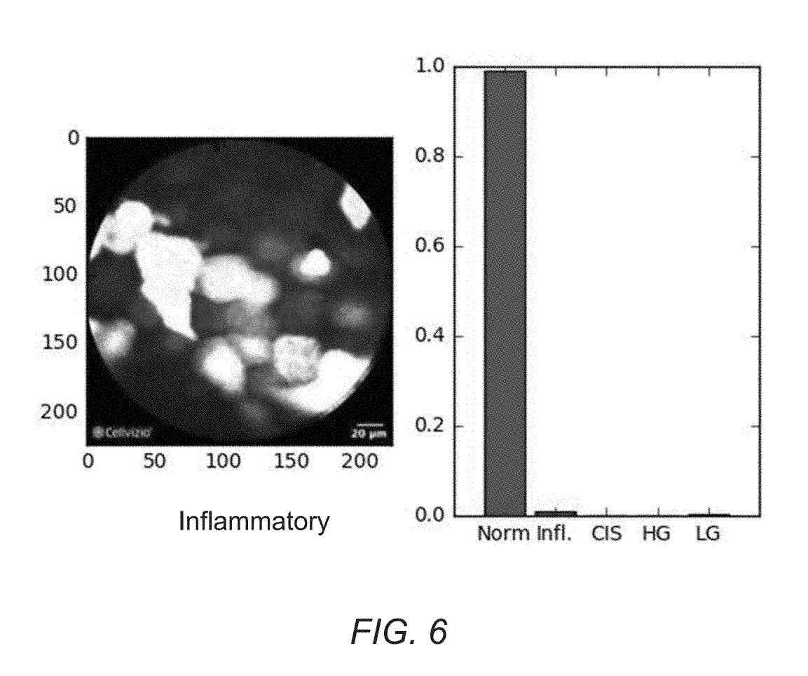

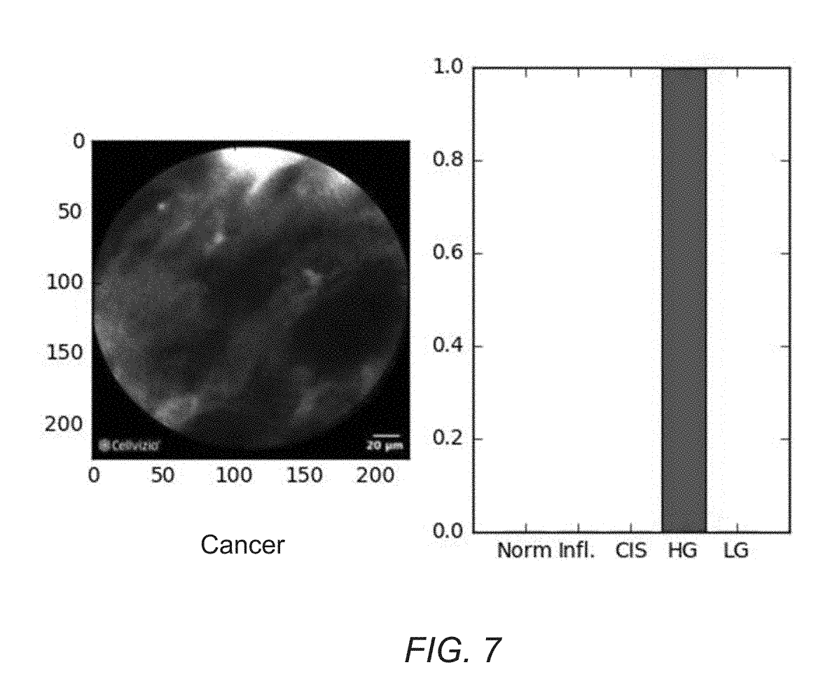

In many embodiments, the disease classification provides histograms indicating the likelihood of the type of cancer for each frame as the frame is provided to the medical professional performing the endoscopy. In this way, the medical professional can take immediate action, such as, but not limited to, more closely observing the area, performing a biopsy, or any other technique as appropriate to the requirements of specific applications of embodiments of the invention. As discussed above, the real-time feedback provided by systems and methods described herein allow medical practitioners to immediately localize where the disease is located within the bladder as the scanning probe will be positioned at or near the identified diseased area when the disease classification is provided. Example disease classifications for an image presenting as normal, inflammatory, and cancerous are illustrated in FIGS. 5, 6, and 7 respectively.

Although certain embodiments of the invention have been described above with respect to the classification of features in CLE images, it may be contemplated that one or more systems and methods of automatic classification as discussed above may be utilized to identify, categorize, evaluate, and/or otherwise provide information regarding features within medical imaging data from various modalities. These modalities may include, but are not limited to, ultrasound and various forms of endoscopy such as optical biopsy technologies, endomicroscopy, probe-based CLE, and other endoscopic technologies.

Although the present invention has been described in certain specific aspects, many additional modifications and variations would be apparent to those skilled in the art. It is therefore to be understood that the present invention can be practiced otherwise than specifically described without departing from the scope and spirit of the present invention. For example, in many embodiments, processes similar to those described above can be performed in differing orders, exclude certain steps, or perform additional steps. Thus, embodiments of the present invention should be considered in all respects as illustrative and not restrictive. Accordingly, the scope of the invention should be determined not by the embodiments illustrated, but by the appended claims and their equivalents.

What is claimed is:

1. An imaging system comprising:
at least one processor;
an input/output interface in communication with a medical imaging device;
a display in communication with the processor; and
a memory in communication with the processor, comprising:
image data obtained from a medical imaging device, where the image data describes at least one image describing at least one region of a patient's body; and
an image processing application, where the image processing application directs the processor to:
preprocess the image data;
identify pathological features within the preprocessed image data;
calculate the likelihood that the at least one region described by the at least one image is afflicted by a disease; and
provide a disease classification substantially instantaneously describing the disease and the likelihood of the disease being present in the region via the display.

2. The imaging system of claim 1, wherein the medical imaging device is a confocal laser endoscope.

3. The imaging system of claim 1, wherein the image data describes a video comprising at least two sequential frames describing at least one region of a patient's body.

4. The imaging system of claim 3, wherein the image processing application further directs the processor to:
preprocess a first frame in the video;
identify pathological features within the first preprocessed frame;
calculate the likelihood of disease in the region described by the first frame;
provide a disease classification substantially instantaneously describing the disease and the likelihood of the disease being present in the region via the display;
preprocess a second frame in the video;
identify pathological features within the second preprocessed frame;
calculate the likelihood of disease in the region described by the second frame; and
update the disease classification substantially instantaneously based on the second frame.

5. The imaging system of claim 3, wherein the image processing application further directs the processor to provide a disease classification for the region described by all of the frames in the video via the display.

6. The imaging system of claim 1, wherein the region of the patient's body is the bladder and the disease is a type of bladder disease selected from the group consisting of high grade cancer, low grade cancer, carcinoma in situ, and inflammation.

7. The imaging system of claim 1, wherein to preprocess the image data, the image processing application further directs the processor to:
standardize the resolution of each frame; and
center each frame.

8. The imaging system of claim 1, wherein to identify pathological features within the preprocessed image data, the image processing application further directs the processor to provide images to a convolutional neural network, where the convolutional neural network is trained by providing classified images of diseased features.

9. The imaging system of claim 8, wherein to calculate the likelihood of disease in a region, the image processing application further directs the processor to obtain a probability score from the convolutional neural network describing the likelihood that the convolutional neural network has correctly identified a disease within the frame.

10. The imaging system of claim 8, wherein the pathological features are structural features of bladder cells associated with any of normal cells, high grade cancer cells, low grade cancer cells, carcinoma in situ cells, and inflammatory cells.

11. A method for providing a substantially instantaneous disease classification based on image data comprising obtaining image data from a medical imaging device, where the image data describes at least one image describing at least one region of patient's body using an image processing server system, wherein the image processing server system comprises:
at least one processor;
an input/output interface in communication with the medical imaging device and the processor;
a display in communication with the processor; and
a memory in communication with the processor, where the memory is configured to store the image data;
preprocessing the image data using an image processing server system;
identifying pathological features within the preprocessed image data;
calculating the likelihood that the at least one region described by the at least one image is afflicted by a disease;
providing a disease classification substantially instantaneously describing the disease and the likelihood of the disease being present in the region via the display.

12. The method of claim 11, wherein the medical imaging device is a confocal laser endoscope.

13. The method of claim 11, wherein the image data describes a video comprising at least two sequential frames describing at least one region of a patient's body.

14. The method of claim 13, wherein the disease classification is provided based on a first frame in the at least two sequential frames; and
updating the disease classification based on the second frame.

15. The method of claim 13, further comprising providing a disease classification for the region described by all of the frames in the video via the display.

16. The method of claim 10, wherein the region of the patient's body is the bladder and the disease is a type of bladder disease selected from the group consisting of high grade cancer, low grade cancer, carcinoma in situ, and inflammation.

17. The method of claim 11, wherein preprocessing the image data comprises:
standardizing the resolution of each frame; and
centering each frame.

18. The method of claim 11, wherein identifying pathological features within the preprocessed image data comprises providing images to a convolutional neural network, where the convolutional neural network is trained by providing classified images of diseased features.

19. The method of claim 18, wherein calculating the likelihood of disease in a region comprises obtaining a probability score from the convolutional neural network describing the likelihood that the convolutional neural network has correctly identified a diseased structure within the frame.

20. The method of claim 18, wherein the pathological features are structural features of bladder cells associated with any of normal cells, high grade cancer cells, low grade cancer cells, carcinoma in situ cells, and inflammatory cells.
